(19) United States
(12) Patent Application Publication
(10) Pub. No.: US 2025/0114662 A1
(43) Pub. Date: Apr. 10, 2025

(54) PERSONALIZED FITNESS COACH ON A TELEVISION

(71) Applicant: GOOGLE LLC, Mountain View, CA (US)

(72) Inventor: Anish Kumar Singh, Pune (IN)

(21) Appl. No.: 18/482,164

(22) Filed: Oct. 6, 2023

(51) Int. Cl. A63B 24/00

(52) U.S. Cl. CPC A63B 24/0075; A63B 24/0062

(57) ABSTRACT

According to an aspect, a method may include rendering, by a television application executing on a computing device, a first user interface that provides a fitness goal setup option associated with an account of a user of the television application. The method may include receiving, by the television application, fitness goal criteria for the fitness goal setup option. The method may include sending, by the computing device, the fitness goal criteria to a server computer for use in generating a media content recommendation directed towards achieving the fitness goal criteria. The method may include receiving a sequence of selectable information items that correspond to media content items sourced by a plurality of different media content providers based on the media content recommendation. The method may include displaying, by the television application, a second user interface that includes the sequence of selectable information items.

### BACKGROUND

Users may want to engage with fitness content provided by streaming services. A user may want to watch the fitness content on a large screen television (TV) or a smart TV. The television may be in the home of the user. The user may stream the fitness content to the television following the instructions provided by the fitness content while performing the exercises in the privacy and comfort of their home.

### SUMMARY

In some aspects, the techniques described herein relate to a method including: rendering, by a television application executing on a computing device, a first user interface that provides a fitness goal setup option associated with an account of a user of the television application; receiving, by the television application, fitness goal criteria for the fitness goal setup option; sending, by the computing device, the fitness goal criteria to a server computer for use in generating a media content recommendation directed towards achieving the fitness goal criteria; receiving a sequence of selectable information items that correspond to media content items sourced by a plurality of different media content providers based on the media content recommendation; and displaying, by the television application, a second user interface that includes the sequence of selectable information items.

In some aspects, the techniques described herein relate to a method, further including receiving, by the computing device, information and data from a wellness device, the information and data providing an indication of at least one of a physical activity of the user or a wellness state of the user.

In some aspects, the techniques described herein relate to a method, further including, in response to receiving the information and data from the wellness device, providing the information and data received from the wellness device to the server computer for use along with the fitness goal criteria in generating the media content recommendation directed towards achieving the fitness goal criteria.

In some aspects, the techniques described herein relate to a method, wherein the method further includes: receiving updated information and data from the wellness device; and in response to receiving the updated information and data from the wellness device, providing the updated information and data received from the wellness device to the server computer for use along with the fitness goal criteria in generating an updated media content recommendation directed towards achieving the fitness goal criteria.

In some aspects, the techniques described herein relate to a method, wherein the media content items are classified into at least one category based on the fitness goal criteria; and wherein displaying the second user interface that includes the sequence of selectable information items includes displaying the sequence of selectable information items for the media content recommendation classified into the at least one category in a cluster.

In some aspects, the techniques described herein relate to a method, wherein the television application includes a plurality of tabbed interfaces; and wherein the method further includes: presenting the first user interface in a first tab of the plurality of tabbed interfaces; and presenting the second user interface in a second tab of the plurality of tabbed interfaces.

In some aspects, the techniques described herein relate to a non-transitory computer-readable medium storing executable instructions that when executed by at least one processor of a server computer cause the at least one processor to execute operations, the operations including: receiving, by the server computer and from a mobile computing device, fitness goal criteria associated with an account of a user of a television application; receiving information and data indicative of at least one of a physical activity of the user or a wellness state of the user; generating a media content recommendation directed towards achieving the fitness goal criteria based on the data indicative of at least one of the physical activity of the user or the wellness state of the user, or a combination thereof; selecting media content items from a plurality of media content providers based on the media content recommendation; and sending a sequence of selectable information items that correspond to the media content items for displaying in a user interface of the television application.

In some aspects, the techniques described herein relate to a non-transitory computer-readable medium, further including providing the information and data indicative of the at least one of the physical activity of the user or the wellness state of the user as training data to an artificial intelligence module.

In some aspects, the techniques described herein relate to a non-transitory computer-readable medium, wherein generating the media content recommendation directed towards achieving the fitness goal criteria includes utilizing the artificial intelligence module.

In some aspects, the techniques described herein relate to a non-transitory computer-readable medium, wherein sending the sequence of selectable information items that correspond to the media content items for displaying in the user interface of the television application further includes: categorizing the media content items into a plurality of categories; and for each category, sending a sequence of selectable information items that correspond to the media content items in a respective category for display in a respective cluster in the user interface of the television application.

In some aspects, the techniques described herein relate to a non-transitory computer-readable medium, wherein the plurality of categories are related to plans and activities for achieving the fitness goal criteria.

In some aspects, the techniques described herein relate to a non-transitory computer-readable medium, wherein the information and data indicative of at least one of the physical activity of the user or the wellness state of the user are first information and data; wherein the media content recommendation is a first media content recommendation; wherein the media content items are first media content items; wherein the sequence of selectable information items that correspond to the media content items are a first sequence of selectable information items that correspond to the first media content items; and wherein the operations further include: receiving second information and data indicative of at least one of the physical activity of the user or the wellness state of the user; generating a second media content recommendation directed towards achieving the fitness goal criteria based on the second information and data indicative of at least one of the physical activity of the user or the wellness state of the user; selecting second media content items from the plurality of media content providers based on the second media content recommendation; and providing a second sequence of selectable information items that correspond to the second media content items for displaying in the user interface of the television application.

In some aspects, the techniques described herein relate to a non-transitory computer-readable medium, further including determining whether the second information and data indicative of at least one of the physical activity of the user or the wellness state of the user indicates an improvement in achieving the fitness goal criteria.

In some aspects, the techniques described herein relate to a non-transitory computer-readable medium, further including, based on determining that the second information and data indicative of at least one of the physical activity of the user or the wellness state of the user indicates an improvement in achieving the fitness goal criteria, providing the second information and data indicative of the at least one of the physical activity of the user or the wellness state of the user and the second media content items as training data to an artificial intelligence module.

In some aspects, the techniques described herein relate to a system including: at least one processor; and a non-transitory computer-readable medium storing executable instructions that execute a television application on a computing device, the television application configured to: render a first user interface that provides a fitness goal setup option associated with an account of a user of the television application; receive fitness goal criteria for the fitness goal setup option; cause the computing device to send the fitness goal criteria to a server computer for use in generating a media content recommendation directed towards achieving the fitness goal criteria; receive a sequence of selectable information items that correspond to media content items sourced by a plurality of different media content providers based on the media content recommendation; and display a second user interface that includes the sequence of selectable information items.

In some aspects, the techniques described herein relate to a system, wherein the television application is further configured to receive information and data from a wellness device, the information and data providing an indication of at least one of a physical activity of the user or a wellness state of the user.

In some aspects, the techniques described herein relate to a system, wherein the television application is further configured to, in response to receiving the information and data from the wellness device, provide the information and data received from the wellness device to the server computer for use along with the fitness goal criteria in generating the media content recommendation directed towards achieving the fitness goal criteria.

In some aspects, the techniques described herein relate to a system, wherein the television application is further configured to: receive updated information and data from the wellness device; and in response to receiving the updated information and data from the wellness device, provide the updated information and data received from the wellness device to the server computer for use along with the fitness goal criteria in generating an updated media content recommendation directed towards achieving the fitness goal criteria.

In some aspects, the techniques described herein relate to a system, wherein the media content items are classified into at least one category based on the fitness goal criteria; and wherein displaying the second user interface that includes the sequence of selectable information items includes displaying the sequence of selectable information items for the media content recommendation classified into the at least one category in a cluster.

In some aspects, the techniques described herein relate to a system, wherein the television application includes a plurality of tabbed interfaces; and wherein the television application is further configured to: present the first user interface in a first tab of the plurality of tabbed interfaces; and present the second user interface in a second tab of the plurality of tabbed interfaces.

### BRIEF DESCRIPTION OF THE DRA WINGS

The details of one or more implementations are set forth in the accompanying drawings and the description below. Other features will be apparent from the description and drawings, and from the claims.

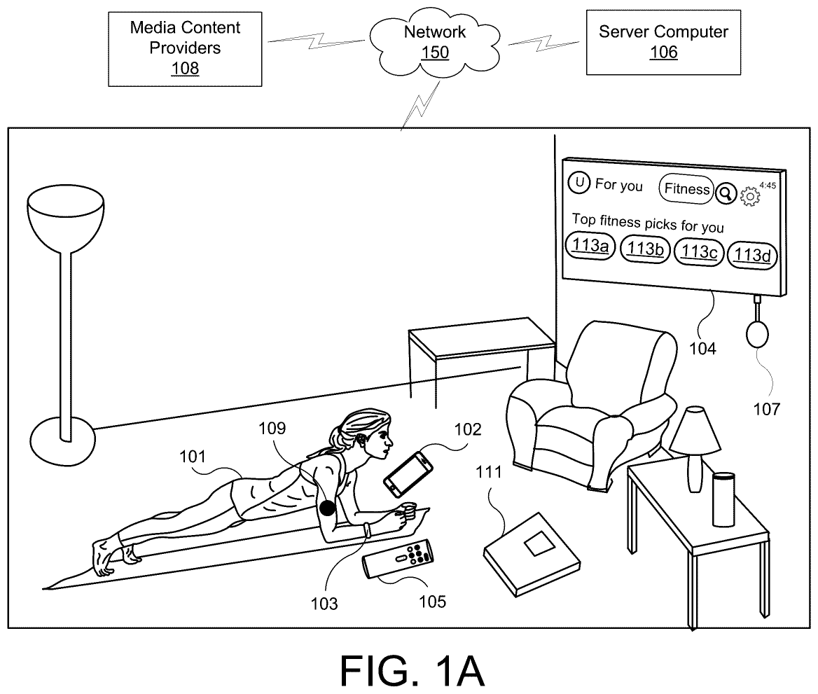

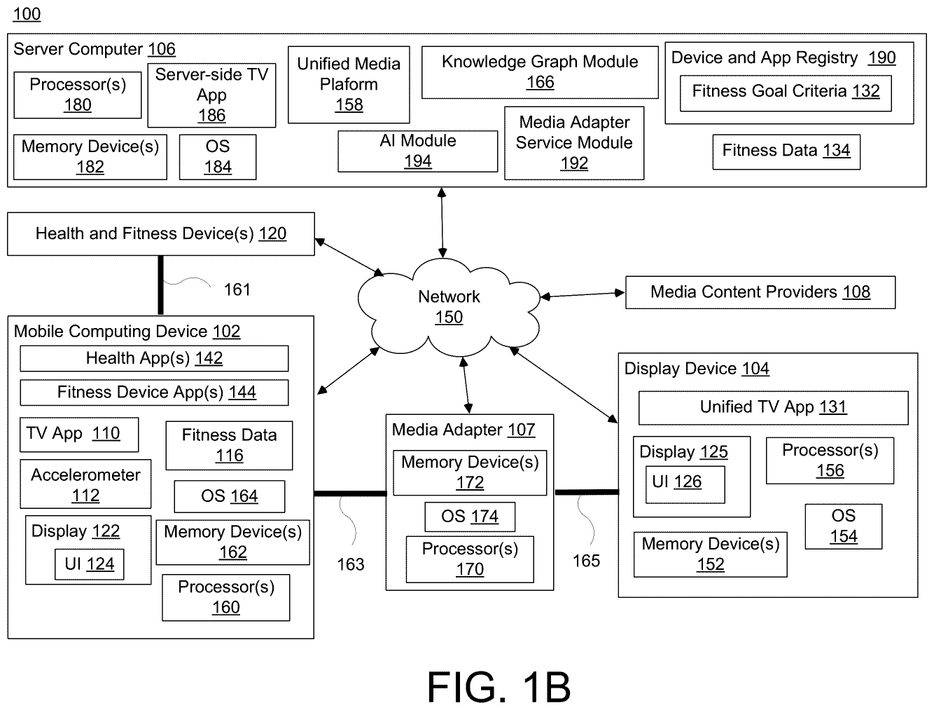

FIG. 1A illustrates an example of a user interacting with a network-connected display device and a media adapter, according to implementations described throughout this disclosure.

FIG. 1B illustrates an example system for gathering fitness and health data, setting fitness goals, and providing personalized fitness recommendations, according to implementations described throughout this disclosure.

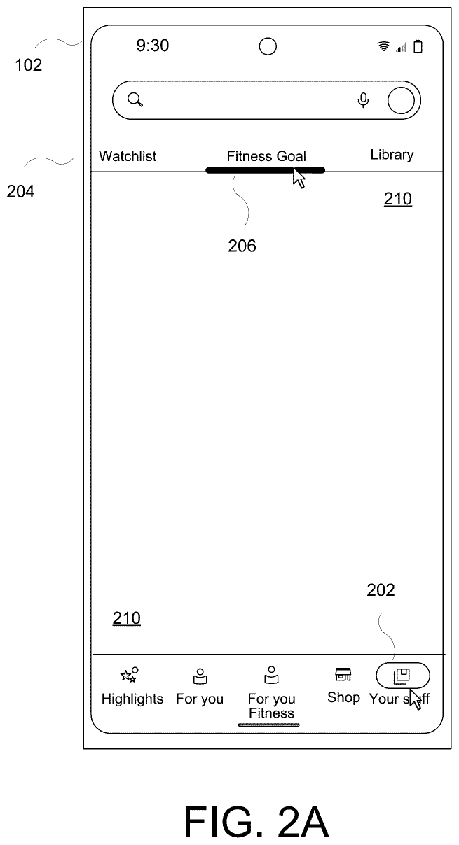

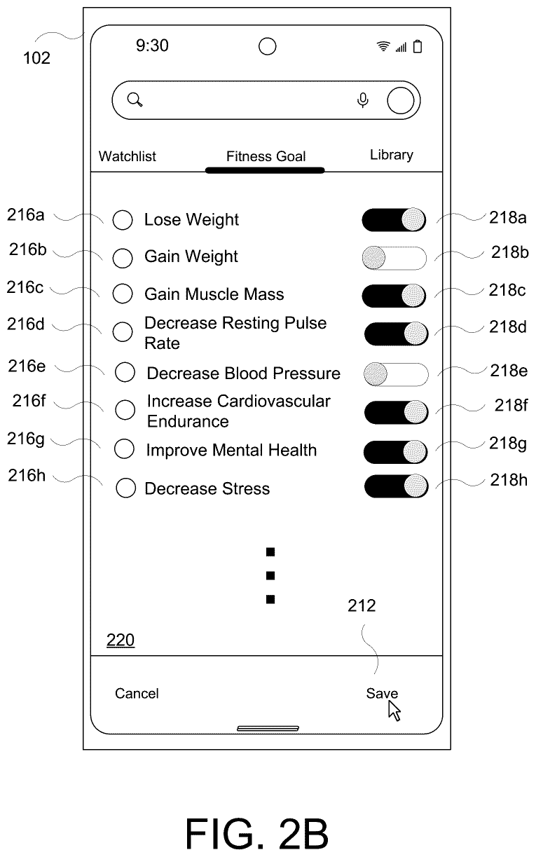

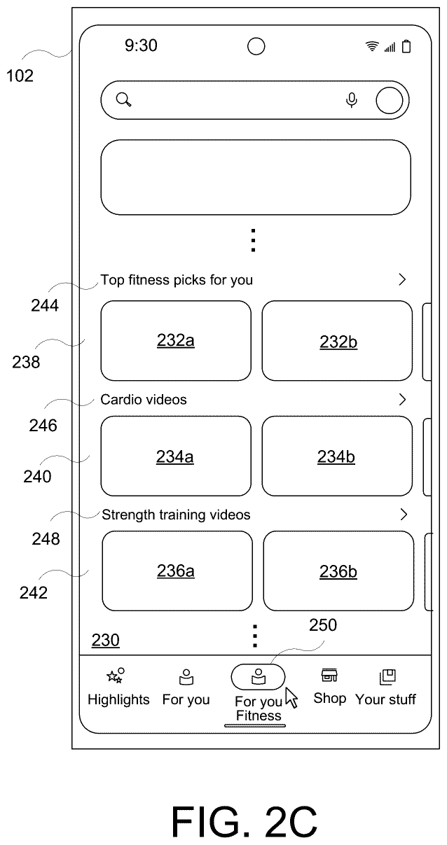

FIGS. 2A-C illustrate example user interfaces for interacting with a television application on a mobile computing device to enter fitness goals and receive personalized fitness recommendations, according to implementations described throughout this disclosure.

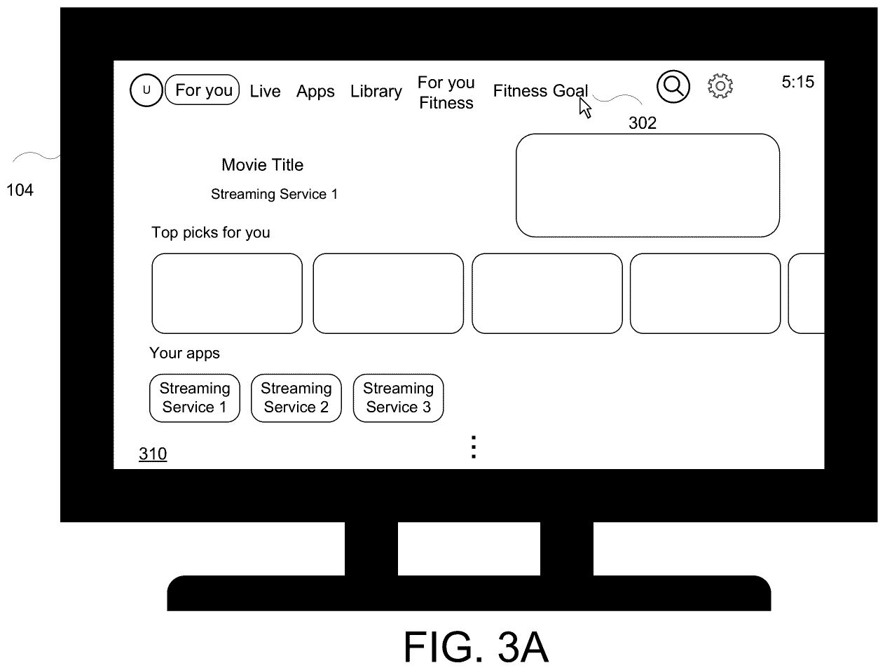

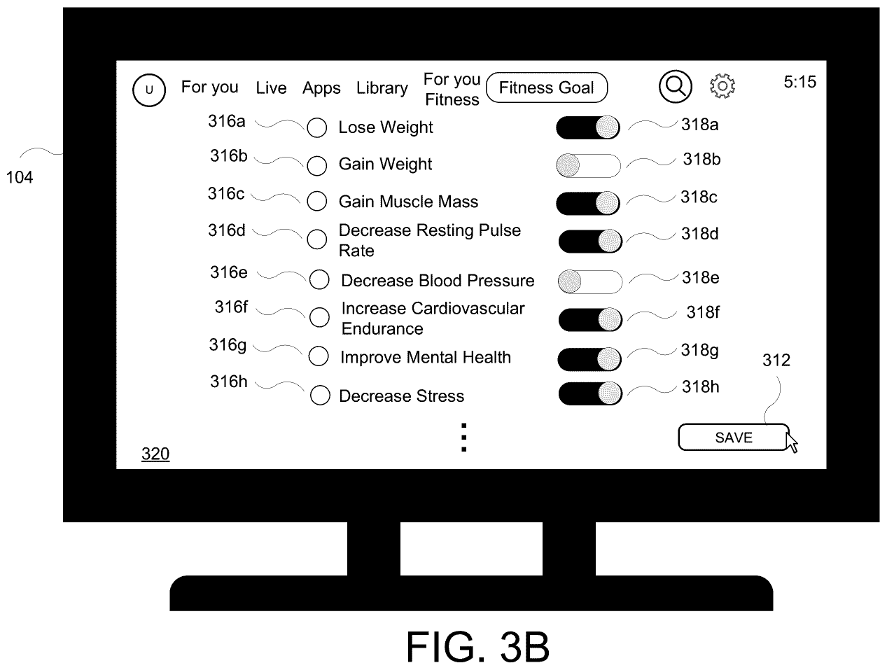

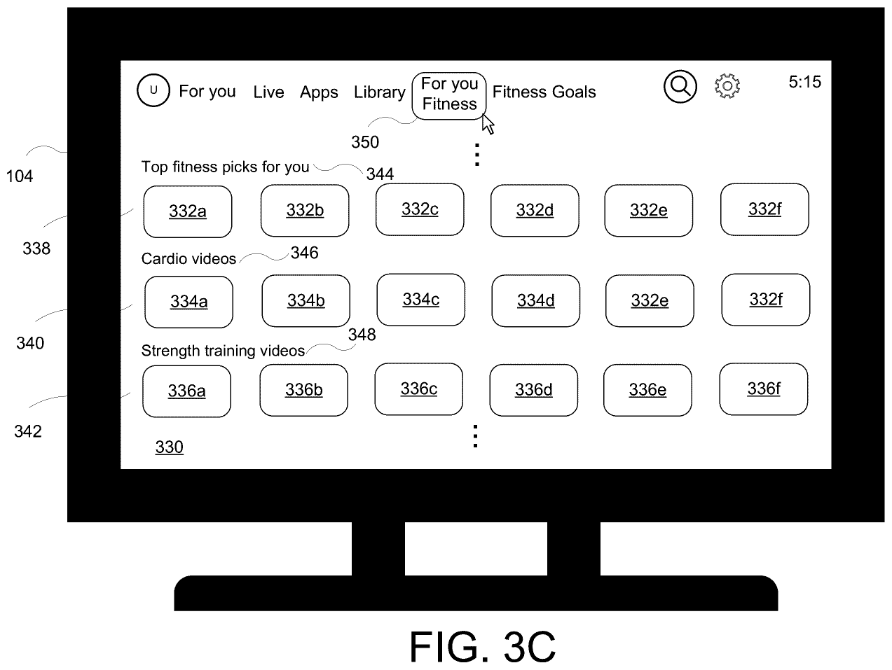

FIGS. 3A-C illustrate example user interfaces for interacting with a television application on a network-connected display device to enter fitness goals and receive personalized fitness recommendations, according to implementations described throughout this disclosure.

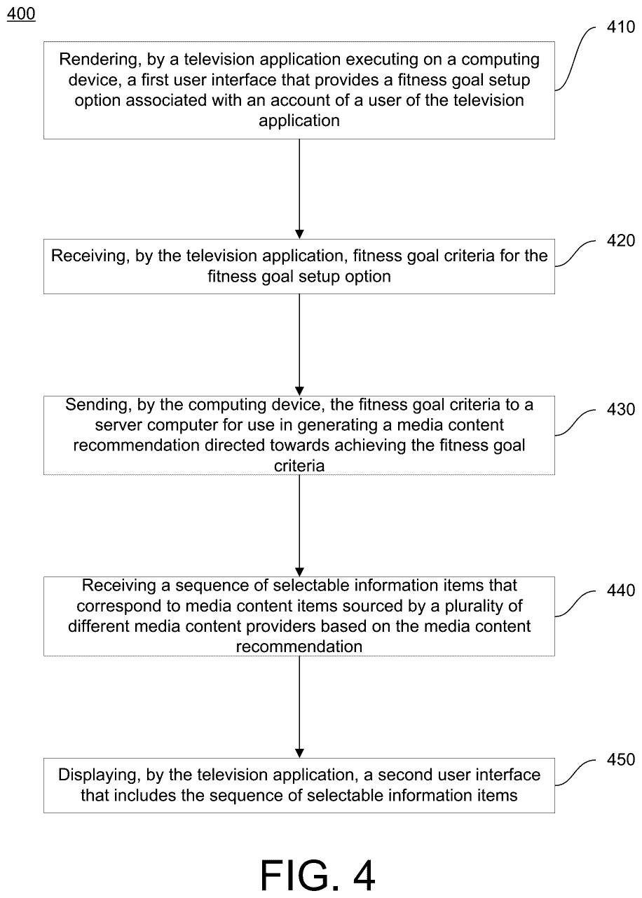

FIG. 4 illustrates a flowchart depicting example operations of gathering fitness and health data, setting fitness goals, and providing personalized fitness recommendations.

### DETAILED DESCRIPTION

In general, a streaming service may provide fitness content to a user that is tailored for a general audience. A streaming service may recommend fitness content to the user based on criteria relevant to a general population that may include, but is not limited to, the popularity of the fitness content or past search history for the fitness content. While the fitness content recommended to the user may be of interest to the user, the recommended fitness content may provide generic fitness advice or exercises for the user. It may be beneficial, however, if a user could interact with an application that could recommend personalized fitness content based on criteria associated with the user, such as for example, health parameters, fitness goals, health improvement goals, weight-loss goals, etc.

A user may want to establish a personalized fitness plan. The fitness plan may include, but is not limited to, an exercise plan, a weight-loss plan, a muscle building plan, a mental health improvement plan, a time management plan, etc. Though streaming services may recommend generalized fitness content and health-related content for the user, the recommended content may not be based on the personal goals of the user. The technical problem is how to provide personalized fitness content recommendations to a user where the fitness content may be media content sourced by different media content providers.

As a technical solution, a television application may provide a fitness goal setup option that allows a user to specify personalized fitness goals (e.g., fitness goal criteria). The television application may gather information and data from wellness devices of the user. As a technical effect, the television application may provide the gathered information and data (e.g., fitness data) for the user along with the fitness goal criteria to an artificial intelligence module for use in model building for determining media content recommendations directed at achieving the fitness goal criteria.

An application on a mobile computing device of a user may include the functionality to provide personalized fitness content recommendations. In some implementations, the application may be a standalone application. In some implementations, the application may be a TV application. The personalized fitness content recommendations may be based on user preferences for health and fitness goals that may include, but are not limited to, weight loss, weight gain, muscle gain, cardiovascular improvements, mental health improvements, etc. The application may provide an interface for the user to enter health and fitness goals that may include, but are not limited to, losing weight, building muscle mass, gaining weight, improving mental health, improving cardiovascular capabilities, increasing a daily step count, decreasing a resting pulse rate, etc.

The application may gather data for the user to apply towards assisting the user in achieving the health and fitness goals of the user. The application may gather data from various devices of the user and other applications that the user may use to gather health and fitness data that may include, but are not limited to, fitness trackers, fitness applications, smart scales, and glucose meters.

The health and fitness data may be gathered or collected on a continuous basis. The application may apply the health and fitness data to the user preferences for health and fitness goals. The application may update the progress of the user towards achieving the health and fitness goals of the user and may provide the progress information to the user. For example, based on the progress information, the user may select different health and fitness content if the user is not achieving the health and fitness goals of the user. In another example, based on the progress information, the user may select similar health and fitness content if the user is working towards achieving the health and fitness goals of the user.

A generative artificial intelligence (generative AI) back end of the application may provide fitness content recommendations that may be categorized and personalized for the user. For example, the generative AI back end may generate health and fitness content recommendations. The application may classify the recommended health and fitness content into categories for each of the user preferences for health and fitness goals. For example, fitness content related to dieting may be placed into or classified in a diet plan category to help achieve a weight loss goal for the user or a weight gain goal for the user. In another example, the application may place or classify the fitness content related to meditation techniques into a mental health category to help achieve a mental health improvement goal for the user.

The application may periodically provide the user preferences for health and fitness goals and the health and fitness data gathered for the user to the generative AI back end of the application. The generative AI backend may use the user preferences and health and fitness data as training data for the generative AI model. For example, the application may keep a record of the progress of the user towards achieving the health and fitness goals of the user. The application may keep a record of the health and fitness content viewed by the user. The application may keep a record of the health and fitness data gathered from the various devices and applications of the user. The application may keep a record of the recommended health and fitness content viewed by the user. Improvements in the health and fitness data gathered from the various devices and applications of the user may indicate that the health and fitness content viewed by the user is successful in helping the user achieve the health and fitness goals of the user. As such, this training data may result in the generative AI back end continuing to recommend the health and fitness content to the user and to other users with similar health and fitness goals.

The generative AI backend may recommend other health and fitness content to the user that is similar to the health and fitness content that was successful in helping the user achieve the health and fitness goals of the user. Conversely, if the health and fitness data gathered from the various devices and applications of the user indicates that the health and fitness content viewed by the user was not successful in helping the user achieve the health and fitness goals of the user, this training data may result in the generative AI back end not continuing to recommend the health and fitness content to the user and to other users with similar health and fitness goals. The generative AI backend may not recommend other health and fitness content to the user that is similar to the health and fitness content that was not successful in helping the user achieve the health and fitness goals of the user.

In implementations where the application is a TV application, the functionality to provide personalized fitness content recommendations may be implemented in a tab of a plurality of tabbed interfaces of the TV application. In these implementations, the TV application may interface with a smart TV (a large screen TV) in the home of the user. The TV application may control the providing of the recommended streaming health and fitness content to the smart TV. The user may watch and interact with the health and fitness content to work towards achieving their health and fitness goals.

FIG. 1A illustrates an example of a user 101 interacting with a network-connected display device 104 and a media adapter 107, according to implementations described throughout this disclosure. FIG. 1A will further be described with reference to FIG. 1B, which is described in more detail herein.

In some implementations, the user 101 may connect to and interact with the media adapter 107 using a television (TV) application 110 installed on a mobile computing device 102. The user, interacting with the TV application 110, may select fitness goal criteria that they want to achieve as part of an overall health and fitness plan. An example of the selection of the fitness goal criteria in this manner will be described in more detail with reference to FIGS. 2A-B.

In some implementations, the user 101 may connect to and interact with the media adapter 107 by way of the network-connected display device 104 using a server-side television (TV) application 186 installed on a server computer 106. The media adapter 107 may be connected or interfaced to the network-connected display device 104. The network-connected display device 104 may be communicatively coupled or connected to the server computer 106 by way of a network 150. The network-connected display device 104 may execute the server-side TV application 186 allowing the user 101 to select fitness goal criteria as shown, for example, with reference to FIGS. 3A-B.

In some implementations, the user 101 may interact with the server-side TV application 186 executing on the network-connected display device 104 using a remote control device 105. In some implementations, the TV application 110 may render a virtual remote control in a user interface. The user may interact with the remote control device 105 and/or the virtual remote control when selecting fitness goal criteria.

Health and fitness device(s) 120 (e.g., wellness devices) may provide health and fitness information and data for the user 101 to the mobile computing device 102. The health and fitness information and data may provide an indication of a physical activity of the user 101. The health and fitness information and data may provide an indication of a wellness state of the user. Examples of the health and fitness device(s) 120 may include, but are not limited to, a fitness tracker 103, a glucose monitor 109, and a smart scale 111. For example, the fitness tracker 103 may provide a number of steps taken by the user, a heart rate of the user, a pulse rate of the user, etc. The glucose monitor 109 may provide continuous glucose monitoring for the user. The smart scale 111 may provide measurements of a weight of a user and in some implementations, may provide information and data related to a body composition for the user such as a body fat percentage, a muscle mass, and a bone mass. In some implementations, the mobile computing device 102 may include an accelerometer 112 that may detect movement of the user for use by health application(s) 142 included on the mobile computing device 102. In this implementation, the mobile computing device 102 may be considered a wellness device. The health and fitness device(s) 120 may be referred to as wellness devices.

The mobile computing device 102 may store the health and fitness information and data received from the health and fitness device(s) 120 in a fitness data repository 116. The mobile computing device 102 may provide the health and fitness information and data to the server computer 106 by way of the network 150. The server computer 106 may use the health and fitness information and data, and the fitness goal criteria to generate media content recommendations for the user. The recommended media content may include media content items or programs based on the fitness goal criteria and the health and fitness information and data. The server computer may curate the recommended media content from media content providers 108. The recommended media content may include streaming media content sourced by the plurality of different media content providers that may help the user achieve one or more of the fitness goal criteria. The server-side TV application 186 may provide a media content recommendation stream for the recommended media content that includes a sequence of selectable information items 113a-d that correspond, respectively, to a plurality of media content items or programs. In some implementations, the server computer may determine an entitlement of the user 101 to access the recommended media content sourced by the media content providers 108 when generating the media content recommendation stream.

For example, the user 101 may select an information item (e.g., information item 113a) using the remote control device 105. The user 101 may watch or interact with the media content associated with the information item 113a as a way of working towards achieving a health and fitness goal for the user 101. For example, the user 101 may watch and participate in a video for a yoga class to help achieve fitness goals for the user 101. In another example, the user may watch and follow a video demonstration of a recipe for a healthy meal. In another example, the user 101 may watch a meditation video to help reduce stress. In some implementations, the TV application 110 may keep a record of the interactions of the user with the media content in the media content recommendation stream. In some implementations, the unified television application 131 may keep a record of the interactions of the user with the media content in the media content recommendation stream.

FIG. 1B illustrates an example system 100 for gathering fitness and health data, setting fitness goals, and providing personalized health and fitness recommendations, according to implementations described throughout this disclosure. The system 100 may include the media content providers 108, the network 150, health and fitness device(s) 120, the mobile computing device 102, the media adapter 107, and the network-connected display device 104. Referring also to FIG. 1A, a user (e.g., the user 101) may interact with the TV application 110 on the mobile computing device 102, the network-connected display device 104, and the server-side TV application 186 to set fitness goals and to receive personalized health and fitness media content recommendations.

For example, the health and fitness device(s) 120 may connect to and interface with the mobile computing device 102. In some implementations, the mobile computing device 102 may connect to and interface with the health and fitness device(s) 120 by way of the network 150. In some implementations the mobile computing device 102 may establish a wireless communication link 161 with each of the health and fitness device(s) 120.

The mobile computing device 102 may be configured to execute at least one health application(s) 142. The mobile computing device 102 may be configured to execute at least one fitness device application(s) 144. In some implementations, the fitness device application(s) 144 may be associated with respective health and fitness device(s) 120. In some implementations, the health application(s) 142 may be associated with respective health and fitness device(s) 120. The fitness device application(s) 144 and the health application(s) 142 may interface with the respective health and fitness device(s) 120 to obtain information and data from the health and fitness device(s) 120 for storage in the fitness data repository 116. In some implementations, the mobile computing device 102 may send or transmit the obtained information and data to the server computer 106 by way the network 150 for storage in a server-side fitness data repository 134.

The mobile computing device 102 may be configured to execute the TV application 110. The mobile computing device 102 includes a mobile computing device display 122 configured to display a user interface (UI) 124. In some implementations, the user may interact with the UI 124 to set up fitness goal criteria in the TV application 110. For example, referring to FIGS. 2A-B which will be described in more detail herein, the TV application 110 may generate one or more user interfaces for use in setting up the fitness goal criteria. In some implementations, referring to FIG. 2C which will be described in more detail herein, the TV application 110 may generate a user interface that includes a sequence of selectable information items that correspond to a plurality of recommended and, in some implementations, categorized media content items directed towards fulfilling the fitness goal criteria of the user. In some implementations, the selection of a selectable information item may cause the TV application 110 to obtain the respective media content from the provider of the media content for viewing on the mobile computing device display 122.

The mobile computing device 102 may be any type of computing device that includes one or more processors (processor(s) 160), one or more memory devices (memory device(s) 162), and an operating system 164. The mobile computing device 102 may be a smartphone, a tablet, a wearable device, a laptop computer, or a desktop computer. In some implementations, the operating system 164 may be system software that manages computer hardware, software resources, and provides common services for computing programs.

In some implementations, the mobile computing device 102 may be a tablet, a smartphone, or a wearable. In these implementations, the operating system 164 may be referred to as a mobile operating system. The mobile operating system may be configured to execute on devices that, in general, include display devices that may be smaller in size than, for example, a display device included in a laptop computer or a desktop computer. In some implementations, the mobile computing device 102 may be a laptop computer. In these implementations, the operating system may be referred to as a laptop or desktop operating system. In these implementations, the operating system 164 may be an operating system designed for a display that is larger in size than that included in a tablet, a smartphone, or a wearable.

The system 100 may include the media adapter 107. The media adapter 107 may facilitate providing (e.g., streaming) media content (e.g., streaming video such as exercise videos, etc.) from one or more streaming services of the media content providers 108 to the network-connected display device 104. For example, the media adapter 107 may directly connect to a connector on the network-connected display device 104 by way of the connection 165. The media adapter 107 may provide digital video and/or audio to the network-connected display device 104. For example, the media adapter 107 may connect to a high-definition multimedia interface (HDMI) connector included in the network-connected display device 104. Examples of the media adapter 107 may include, but are not limited to, a set-top box, a television box, and a streaming media adapter.

The media adapter 107 may be configured to stream media content received from the media content providers 108 by way of the network 150 to the network-connected display device 104. In a non-limiting example, the media adapter 107 may be a casting device and the network-connected display device 104 may be a television (e.g., a smart TV).

The media adapter 107 may be any type of computing device that includes one or more processors (processor(s) 170), one or more memory devices (memory device(s) 172), and an operating system 174. In some implementations, the processor(s) 170 may include a system on a chip (SoC). The SoC may include a central processing unit (CPU), a graphic processing unit (GPU), one or more memory interfaces, and one or more input/output interfaces and devices. In some implementations, the operating system 174 may be system software that manages computer hardware, software resources, and provides common services for computing programs.

The system 100 may include the network-connected display device 104. The network-connected display device 104 may include a display 125. The network-connected display device 104 may display a user interface (UI) 126 on the display 125. In some implementations, referring to FIG. 3C which will be described in more detail herein, the server-side TV application 186 may generate a sequence of selectable information items that correspond to a plurality of recommended and, in some implementations, categorized media content items directed towards fulfilling the fitness goal criteria of the user. The sequence of selectable information items may be referred to as a media content recommendation stream. The server computer 106 may transmit or send the sequence of selectable information items to the media adapter 107 for display in the UI 126.

Once the media adapter 107 receives an indication of the selection of the information item, the media adapter 107 may request the media content item associated with the information item from the media content provider sourcing the media content item. The media adapter 107 may provide the media content (e.g., stream the media content) received from the media content provider to the network-connected display device 104 for viewing on the display 125.

In some implementations, the network-connected display device 104 may include a unified television application 131. For example, the network-connected display device 104 may be a smart television. For example, a smart television may be a network-enabled television that may connect to media content providers by way of a network. The media content providers may source media content to the smart television. In these implementations, a user may interact with the unified television application 131 to access media content from the media content providers 108. The unified television application 131 may interface with the server computer 106, and specifically with the server-side TV application 186. The unified television application 131 may provide similar functionality to the user as that provided by an application executing on the media adapter 107. For example, executing the unified television application 131 by the network-connected display device 104 allows the network-connected display device 104 to obtain a media content recommendation stream from the server computer 106.

The network-connected display device 104 may be configured to connect to the network 150. In some implementations, the network-connected display device 104 is a television (e.g., a smart television (TV)). The network-connected display device 104 may include one or more processors (processor(s) 156), one or more memory devices (memory device(s) 152), and an operating system (OS) 154. The operating system 154 may execute (or assist with executing) the unified television application 131.

In some implementations, the operating system 154 may be a browser application. A browser application is a web browser configured to access information on the Internet by way of a network (e.g., network 150). A browser application may launch one or more browser tabs in the context of one or more browser windows in the browser application. In some implementations, the operating system 154 is a Linux-based operating system configured to execute (or assist with executing) the unified television application 131.

The system 100 includes one or more server computers (e.g., the server computer 106) configured to interface with the mobile computing device 102, the health and fitness device(s) 120, the media adapter 107, the media content providers 108, and the network-connected display device 104 by way of the network 150. In some implementations, the network 150 may establish a wireless communication link between the network-connected display device 104, the mobile computing device 102, the media adapter 107, the media content providers 108, the health and fitness device(s) 120, and the server computer 106.

The server computer 106 may include a unified media platform (UMP) 158. The UMP 158 may manage media content recommendations. The UMP 158 may manage the providing of the media content associated with the media content recommendations from the media content providers 108 to the network-connected display device 104. In some implementations, the UMP 158 may manage the providing of the media content associated with the media content recommendations from the media content providers 108 to the mobile computing device 102. The UMP 158 may provide the media content recommendation stream to the network-connected display device 104. The UMP 158 may provide the media content recommendation stream to the media adapter 107, which in turn streams the selected media content from the media content providers 108 to the network-connected display device 104. In some implementations, the server-side television application 186 may provide the media content recommendation stream for presenting on the network-connected display device 104 by the unified television application 131. In response to receiving an indication of a selection from the media content recommendations, the server-side television application 186 may enable display of the media content on the display 125. In some implementations, the UMP 158 may function as a centralized media content management module configured to provide the media content recommendations to the mobile computing device 102.

The server computer 106 may include a knowledge module 166. The knowledge module 166 may generate media content recommendations for associating with an account of a user based, in part, on a multi-dimensional user activity characteristic associated with the account of the user and the information associated with media content items provided by the media content providers 108. The user activity characteristic associated with the account of the user may be obtained from a plurality of information sources that may include, but are not limited to, a search engine, a mapping application, and an online retailer. The information sources may provide activity data related to activities of the account of the user by way of a respective software program or application.

The knowledge module 166 may generate media content recommendations for associating with an account of a user further based, in part, on knowledge of the user (e.g., interests and activities) as determined from the interactions of the user with the TV application 110, the media adapter 107, and the network-connected display device 104. For example, the account of the user associated with the mobile computing device 102, the media adapter 107, or the network-connected display device 104 may be associated with one or more information sources (e.g., applications of the user).

In some implementations, the server computer 106 may include a device and application registry 190. The device and application registry 190 may be configured to store information and data for an account of a user that is managed by the server computer 106. The device and application registry 190 may be configured to store information and data for device(s) of the user. The device and application registry 190 may be configured to store information and data for programs and applications associated with the account of the user. The device and application registry 190 may store activity data of the programs and applications of the user in association with the account of the user. The device and application registry 190 may store activity data of the use of the device(s) of the user. For example, device and application registry 190 may store fitness goal criteria in a fitness goal criteria repository 132. The device and application registry 190 may store fitness data in a server-side fitness data repository 134. The device and application registry 190 may also store information of the network-connected display device 104, the media adapter 107, and the mobile computing device 102.

In some implementations, the UMP 158 may gather or collect activity information and data associated with the account of the user from the server-side TV application 186. The UMP 158 may gather or collect activity information and data associated with the account of the user from a media adapter service module 192. The media adapter service module 192 may receive a search query or request for media content from the media adapter 107. The UMP 158 may identify one or more media content items from the program information of programs or content items provided by the media content providers 108. The UMP 158 may provide the program information to the knowledge module 166 to update the unified user activity characteristic and media content recommendations associated with the account of the user.

The server computer 106 may regularly (e.g., once a minute, hourly, daily, weekly, monthly, etc.) receive the fitness data from the fitness data repository 116 for storage in the server-side fitness data repository 134. The server computer 106 may regularly (e.g., once a minute, hourly, daily, weekly, monthly, etc.) receive the fitness goal criteria from the TV application 110 for storage in the fitness goal criteria repository 132. The server computer 106 may regularly (e.g., once a minute, hourly, daily, weekly, monthly, etc.) receive the fitness goal criteria from the server-side TV application 186 as gathered by the unified television application 131 executing on the network-connected display device 104 for storage in the server-side fitness data repository 134. The UMP 158 may coordinate the updating of the fitness data in both the server-side fitness data repository 134 and the fitness data repository 116.

The server computer 106 may include an artificial intelligence (AI) module 194. The AI module 194 may receive fitness goal criteria for a user from the fitness goal criteria repository 132. The AI module 194 may receive fitness data for the user from the server-side fitness data repository 134. The AI module 194 may receive information and data from the knowledge module 166, such as the media content recommendations associated with an account of the user. The AI module 194 may use the fitness goal criteria, the fitness data, and the media content recommendations to build an AI model for use by the AI module 194. In some implementations, the AI module 194 may provide the server-side TV application 186 with a plurality of recommended and, in some implementations, categorized media content items directed towards fulfilling the fitness goal criteria of the user that is based on the fitness data for the user and the media content preferences for the user as determined by the knowledge module 166. The server-side TV application 186 may generate the media content recommendation stream as a sequence of selectable information items that correspond to the plurality of recommended and categorized media content items.

The AI module 194 may receive updated media content recommendations from the knowledge module 166 along with updated fitness data for the user and/or updated fitness goal criteria for the user to retrain the AI model. The AI module 194 may use the retrained AI model to provide the server-side TV application 186 with an updated plurality of recommended and, in some implementations, categorized media content items directed towards fulfilling the updated fitness goal criteria of the user that is based on the updated fitness data for the user and the updated media content preferences for the user as determined by the knowledge module 166. The server-side TV application 186 may generate the updated media content recommendation stream as a sequence of updated selectable information items that correspond to the updated plurality of recommended and categorized media content items.

In some implementations, the AI module 194 may use generative artificial intelligence along with a generative AI model. For example, in these implementations, the AI module 194 may be referred to as a generative artificial intelligence (generative AI) back end. For example, generative AI may identify themes related to media content viewed by the user across multiple different media content providers. The generative AI may curate or create a collection of media content recommendations based on the identified themes. As such, the use of generative AI may provide the user with customized media content recommendations that may be regularly modified and updated based on changes in the fitness goal criteria, the fitness data, and the viewing habits of the user as identified by the knowledge module 166.

In some implementations, the TV application 110 may keep a record of the interactions of the user with the media content in the media content recommendation stream. For example, the TV application 110 may store this information in the memory device(s) 162 included in the mobile computing device 102. The mobile computing device 102 may send or transmit the usage record to the server computer 106 for use by the AI module 194 when recommending media content to the user. In some implementations, the unified television application 131 may keep a record of the interactions of the user with the media content in the media content recommendation stream. For example, the unified television application 131 may store this information in the memory device(s) 172. The media adapter 107 may send or transmit the usage record to the server computer 106 for use by the AI module 194 when recommending media content to the user. In another example, the network-connected display device 104 may be a smart television. The unified television application 131 may store this information in the memory device(s) 152. The network-connected display device 104 may send or transmit the usage record to the server computer 106 for use by the AI module 194 when recommending media content to the user.

In some implementations, generative AI may generate text for a message to the user. For example, the AI module 194 may receive updated media content recommendations from the knowledge module 166 along with updated fitness data for the user and/or updated fitness goal criteria for the user to retrain the AI model. The AI module 194 may use the retrained AI model to provide the server-side TV application 186 with an updated plurality of recommended and, in some implementations, categorized media content items directed towards fulfilling the updated fitness goal criteria of the user that is based on the updated fitness data for the user and the updated media content preferences for the user as determined by the knowledge module 166. Based on the updated information and data, the AI module 194 may determine that the user may not have achieved the original fitness goal criteria for the user. For example, the AI module 194 may determine that the user interacted very little if at all with the previously recommended media content. For example, the use of generative AI may enable the AI module 194 to generate text for a message to the user that may inspire the user to interact more with the recommended media content items (e.g., “We know that it can be hard to get started, but a small amount of exercise can make a big difference. Here are some recommendations. Have fun!”:

In some implementations, the server computer 106 can transmit or send the text for the message to the mobile computing device 102. For example, the TV application 110 may display the text for the message in the UI 124. In another example, the mobile computing device 102 may provide text-to-voice messaging and may convert the text of the message to an audible voice message that may be provided to the user. In some implementations, the server computer 106 can transmit or send the text for the message to the network-connected display device 104. For example, the unified television application 131 may display the text for the message in the UI 126.

In some implementations, generative AI may generate description and information text related to the recommended media content. The description and information text may be generated based on the received fitness data for the user, the fitness goal criteria for the user, and/or the record of the interactions with any previously recommended media content. In some implementations, the description and information text may be generated based on the category associated with the recommended media content. In some implementations, the description and information text may be generated based on the applicability of the recommended media content to the achievement of fitness goals. For example, the use of generative AI may enable the AI module 194 to generate descriptive text and information directed towards sleep improvement (e.g., “Based on the data from your fitness tracker, we noticed that you have not been able to get quality sleep. Here are some recommended videos on meditation that may help you achieve better sleep.”).

In some implementations, the server computer 106 can transmit or send the description and information text to the mobile computing device 102. For example, referring to FIG. 2C, the TV application 110 may display the description and information text along with the descriptive text for the category associated with the recommended media content. In some implementations, the server computer 106 can transmit or send the description and information text to the network-connected display device 104. For example, referring to FIG. 3C, the unified television application 131 may display the description and information text along with the descriptive text for the category associated with the recommended media content.

The system 100 includes the media content providers 108. The media content providers 108 may source media content items to the media adapter 107 for viewing on the network-connected display device 104. In some implementations, the media content providers 108 may source media content items to the network-connected display device 104 for viewing as the media content on the display 125. In some implementations, the media content providers 108 may source media content items to the mobile computing device 102 for viewing on the mobile computing device display 122. In some implementations, the media content providers 108 may include subscription-based streaming services and/or no-fee based streaming services. In some implementations, the UMP 158 may determine an entitlement of a user to access and view content sourced by a subscription-based streaming service included in the media content providers 108. The UMP 158 may make this determination before providing a media content recommendation sourced by a subscription-based streaming service that the user is not entitled to access.

The server computer 106 may be computing devices that take the form of a number of different devices, for example a standard server, a group of such servers, or a rack server system. In some implementations, the server computer 106 may be a single system sharing components such as one or more processors (e.g., processor(s) 180) and one or more memory devices (e.g., memory device(s) 182).

The mobile computing device 102 may include the mobile computing device display 122. In some implementations, the mobile computing device display 122 is a display device such as a liquid crystal display (LCD), a light-emitting diode (LED) display, an organic light-emitting diode (OLED) display, or an active-matrix organic light-emitting diode (AMOLED) display. The network-connected display device 104 may include the display 125. In some implementations, the display 125 is a display device such as a liquid crystal display (LCD), a light-emitting diode display (LED) display, a plasma display, a quantum dot light-emitting diode display (QLED) display, or an organic light-emitting diode (OLED) display.

The processor(s) 156, the processor(s) 160, the processor(s) 170, and the processor(s) 180 may be formed in a substrate configured to execute one or more machine executable instructions or pieces of software, firmware, or a combination thereof. The processor(s) 156, the processor(s) 160, the processor(s) 170, and the processor(s) 180 may be semiconductor-based. For example, the processor(s) 156, the processor(s) 160, the processor(s) 170, and the processor(s) 180 may include semiconductor material that can perform digital logic.

The memory device(s) 152, the memory device(s) 162, the memory device(s) 172, and the memory device(s) 182 may include main memory that stores information in a format that can be read and/or executed by the processor(s) 156, the processor(s) 160, the processor(s) 170, and the processor(s) 180 respectively. The memory device(s) 152, the memory device(s) 162, the memory device(s) 172, and the memory device(s) 182 may include one or more random-access memory (RAM) devices and/or one or more read-only memory (ROM) devices.

The memory device(s) 152, memory device(s) 162, the memory device(s) 172, and the memory device(s) 182 may store applications that, when executed by the processor(s) 156, the processor(s) 160, the processor(s) 170, and the processor(s) 180, respectively, perform operations. For example, the memory device(s) 162 may store the operating system 164 and the TV application 110 that, when executed by the processor(s) 160, may perform operations on the mobile computing device 102. For example, the memory device(s) 152 may store operating system 154 and the unified television application 131 that, when executed by the processor(s) 156, may perform operations on the network-connected display device 104.

In some implementations, the memory device(s) 182 may represent any kind of (or multiple kinds of) memory (e.g., RAM, flash, cache, disk, tape, etc.). In some implementations, the memory device(s) 182 may include external storage, e.g., memory physically remote from but accessible by the server computer 106. The server computer 106 may include one or more modules, engines, or applications representing specially programmed software. In some implementations, the server computer 106 may include the operating system 184, the server-side TV application 186, the UMP 158, the knowledge module 166, the AI module 194, and the media adapter service module 192. For example, the memory device(s) 182 may store the operating system 184, the server-side TV application 186, the UMP 158, the knowledge module 166, the AI module 194, and the media adapter service module 192 that, when executed by the processor(s) 180, may perform operations on server computer 106 to facilitate providing personalized fitness content recommendations to a user.

The network 150 may include the Internet and/or other types of data networks, such as a local area network (LAN), a wide area network (WAN), a cellular network, satellite network, or other types of data networks. The network 150 may also include any number of computing devices (e.g., computer, servers, routers, network switches, etc.) that are configured to receive and/or transmit data within the network 150. The network 150 may further include any number of hardwired and/or wireless connections. The network 150 may be, for example, communications networks having one or more types of topologies, including but not limited to the Internet, intranets, local area networks (LANs), cellular networks, Ethernet, Storage Area Networks (SANs), telephone networks, and Bluetooth personal area networks (PAN). In some implementations, two or more devices in a sub-network may be coupled by way of a wired connection, while at least some of the devices in the same sub-network are coupled by way of a local radio communication network (e.g., ZigBee. Z-Wave, Insteon, Bluetooth, Wi-Fi and other radio communication networks).

In some implementations, the wireless communication link 161 and the wireless communication link 163 may be short-range wireless connections such as a Bluetooth connection. In some examples, the wireless communication link 161 and the wireless communication link 163 may be a Wi-Fi (e.g., direct Wi-Fi) connection.

FIGS. 2A-C illustrate example user interfaces for interacting with a television application on a mobile computing device to enter fitness goals and receive personalized fitness recommendations, according to implementations described throughout this disclosure.

FIG. 2A illustrates an example initial user interface (e.g., initial user interface 210) for a TV application (e.g., the TV application 110). For example, referring to FIGS. 1A-B, a user may launch the TV application 110 on the mobile computing device 102. The TV application 110 may include a plurality of tabbed interfaces. Upon launching, the TV application 110 may render the initial user interface 210 in a tab of a plurality of tabbed interfaces in the UI 124 on the mobile computing device display 122. The user may select a your stuff option 202 from the initial user interface 210 that will display a selectable list of options 204 for the TV application 110. The selectable list of options 204 includes a fitness goal setup option 206. The user may select the fitness goal setup option 206 when initially setting up fitness goal criteria or when updating existing fitness goal criteria.

FIG. 2B illustrates an example first user interface (e.g., first user interface 220) for a TV application (e.g., the TV application 110). For example, referring to FIGS. 1A-B and FIG. 2A, based on receiving an indication of the selection of the fitness goal setup option 206, the TV application 110 may provide the first user interface 220 in the UI 124 on the mobile computing device display 122. The first user interface 220 may be presented in a first tab of the tabbed interfaces for the user interface for the TV application 110. The first user interface 220 presents a list of fitness goal criteria 216a-h for selection by the user. The list of fitness goal criteria 216a-h is an example list and, in some implementations, may include more, less, and/or different fitness goal criterion. For example, each of the fitness goal criteria 216a-h is associated with a respective sliding button of sliding buttons 218a-h that a user can control to select or deselect (not select) the associated fitness goal criterion. In the example shown in FIG. 2B, the user selected the following fitness goal criteria: lose weight, gain muscle mass, decrease resting pulse rate, increase cardiovascular endurance, improve mental health, and decrease stress. The user may then select a save option 212. The TV application 110 may then send or transmit the fitness goal criteria to the server computer 106 for storage in the fitness goal criteria repository 132.

The use of the sliding buttons 218a-h provides a technical benefit in that it facilitates the collection of binary inputs from the user for the purpose of identifying their fitness goal criteria for provision as input to the AI model at the server computer 106. The use of binary (yes and no options) for the fitness goal criteria simplifies the user's interaction with the TV application 110 for the user. It also simplifies the inputs to the AI model, and thus potentially allows the fitness media recommendations to be provided using fewer resources than with a corresponding arrangement in which each input could have more than two options. Instead of sliding buttons, the first user interface 220 may present a list of fitness goal criteria 316a-h for selection by the user using toggle switches, radio buttons or other binary user interface elements that allow a user to select between yes and no options.

FIG. 2C illustrates an example second user interface (e.g., second user interface 230) for a TV application (e.g., the TV application 110). For example, referring to FIGS. 1A-B and FIG. 2B, based on receiving an indication of the selection of the save option 212, the TV application 110 may provide the second user interface 230 in the UI 124 on the mobile computing device display 122. The second user interface 230 may be presented in a second tab of the tabbed interfaces for the user interface for the TV application 110. In some implementations, the user may select a for you fitness option 250 from the second user interface 230 that causes the display of the second user interface 230 for the TV application 110. The second user interface 230 may provide a sequence of selectable information items such as, for example, first selectable information items 232a-b, second selectable information items 234a-b, and third selectable information items 236a-b. The selectable information items may correspond, respectively, to a plurality of media content items or programs for recommended media content.

In some implementations, the AI module 194 may use the fitness goal criteria stored in the fitness goal criteria repository 132, the fitness data stored in the server-side fitness data repository 134, and the media content recommendations provided by the knowledge module 166 to provide the TV application 110 with a plurality of recommended and categorized media content items directed towards fulfilling the fitness goal criteria of the user based on the fitness data for the user and the media content preferences for the user as determined by the knowledge module 166. In some implementations, the server-side TV application 186 may generate the media content recommendation stream as a sequence of selectable information items (e.g., the first selectable information items 232a-b, the second selectable information items 234a-b, and the third selectable information items 236a-b) for display in a cluster (e.g., a first cluster 238, a second cluster 240, a third cluster 242, respectively) for each category (e.g., a “top fitness picks for you” category 244, a cardio videos category 246, a strength training videos category 248, respectively) that correspond to the plurality of recommended and categorized media content items. The sequence of selectable information items may be referred to as a media content recommendation stream. The server-side TV application 186 may generate the media content recommendation stream. The server-side TV application 186 may cause the server computer 106 to send or transmit the media content recommendation stream to the mobile computing device 102 by way of network 150. The TV application 110 may cause the display of the media content recommendation stream in the UI 124.

Referring to FIG. 2C, the first selectable information items 232a-b are included in a top fitness picks category 244 for the user. For example, the AI module 194 may determine that the media content associated with the first selectable information items 232a-b address the fitness goal criteria for the user considering the fitness data for the user. The second selectable information items 234a-b are included in a cardio videos category 246. For example, the AI module 194 may determine that the media content associated with the second selectable information items 234a-b address the cardio-related fitness goal criteria (e.g., decrease blood pressure, decrease resting pulse rate, increase cardiovascular endurance) for the user considering the fitness data for the user. For example, the AI module 194 may determine that the media content associated with the third selectable information items 236a-b address strength training related fitness goal criteria (e.g., gain muscle mass) for the user considering the fitness data for the user.

FIGS. 3A-C illustrate example user interfaces for interacting with a television application on a network-connected display device to enter fitness goals and receive personalized fitness recommendations, according to implementations described throughout this disclosure.

FIG. 3A illustrates an example initial user interface (e.g., initial user interface 310) for a TV application (e.g., the unified television application 131). For example, referring to FIGS. 1A-B, in some implementations a user may launch the unified television application 131 on the network-connected display device 104. The unified television application 131 may include a plurality of a tabbed interfaces. The unified television application 131 may render the initial user interface 310 in a tab of the plurality of tabbed interfaces in the UI 126 on the network-connected display device 104. In some implementations, the user may interact with an application executing on the media adapter 107 to cause the media adapter 107 to render the initial user interface 310 for displaying on the network-connected display device 104. The user may select a fitness goal setup option 302 from the initial user interface 310. The user may select the fitness goal setup option 206 when initially setting up fitness goal criteria or when updating existing fitness goal criteria.

FIG. 3B illustrates an example first user interface (e.g., first user interface 320) for a TV application (e.g., the unified television application 131). For example, referring to FIGS. 1A-B and FIG. 3A, based on receiving an indication of the selection of the fitness goal setup option 302, the unified television application 131 may render the first user interface 320 in a tab of the tabbed interfaces in the UI 126 on the network-connected display device 104. The first user interface 320 may be presented in a first tab of the user interface for the unified television application 131. The first user interface 320 presents a list of fitness goal criteria 316a-h for selection by the user. The list of fitness goal criteria 316a-h is an example list and, in some implementations, may include more, less, and/or different fitness goal criterion. For example, each of the fitness goal criteria 316a-h is associated with a respective sliding button of sliding buttons 318a-h that a user can control to select or deselect (not select) the associated fitness goal criterion. In the example shown in FIG. 3B, the user selected the following fitness goal criteria: lose weight, gain muscle mass, decrease resting pulse rate, increase cardiovascular endurance, improve mental health, and decrease stress. The user may then select a save button 312 to save the fitness goal criteria in the fitness goal criteria repository 132.

The use of the sliding buttons 318a-h provides a technical benefit in that it facilitates the collection of binary inputs from the user for the purpose of identifying their fitness goal criteria for provision as input to the AI model at the server computer 106. The use of binary (yes and no options) for the fitness goal criteria simplifies the user's interaction with the unified television application 131 for the user. It also simplifies the inputs to the AI model, and thus potentially allows the fitness media recommendations to be provided using fewer resources than with a corresponding arrangement in which each input could have more than two options. Instead of sliding buttons, the first user interface 320 may present a list of fitness goal criteria 316a-h for selection by the user using toggle switches, radio buttons or other binary user interface elements that allow a user to select between yes and no options.

FIG. 3C illustrates an example second user interface (e.g., second user interface 330) for a TV application (e.g., the unified television application 131). For example, referring to FIGS. 1A-B and FIG. 3B, based on receiving an indication of the selection of the save button 312, the unified television application 131 may provide the second user interface 330 in the UI 126 on the display 125. The second user interface 330 may be presented in a second tab of the plurality of tabbed interfaces for the unified television application 131. In some implementations, the user may select a for you fitness option 350 from the second user interface 330 that causes the display of the second user interface 330 for the unified television application 131. The second user interface 330 may provide a sequence of selectable information items such as, for example, first selectable information items 332a-f, second selectable information items 334a-f, and third selectable information items 336a-f. The selectable information items may correspond, respectively, to a plurality of media content items or programs for recommended media content.

In some implementations, the AI module 194 may use the fitness goal criteria stored in the fitness goal criteria repository 132, the fitness data stored in the server-side fitness data repository 134, and the media content recommendations provided by the knowledge module 166 to provide the unified television application 131 with a plurality of recommended and categorized media content items directed towards fulfilling the fitness goal criteria of the user based on the fitness data for the user and the media content preferences for the user as determined by the knowledge module 166. The unified television application 131 may generate a sequence of selectable information items (e.g., the first selectable information items 332a-f, the second selectable information items 334a-f, and the third selectable information items 336a-f) for display in a cluster (e.g., a first cluster 338, a second cluster 340, a third cluster 342, respectively) for each category (e.g., a “top fitness picks for you” category 344, a cardio videos category 346, a strength training videos category 348, respectively) that correspond to the plurality of recommended and categorized media content items. The sequence of selectable information items may be referred to as a media content recommendation stream. The server-side TV application 186 may generate the media content recommendation stream. In some implementations, the server-side TV application 186 may cause the server computer 106 to send or transmit the media content recommendation stream to the network-connected display device 104 by way of network 150. The unified television application 131 may cause the display of the media content recommendation stream in the UI 126. In some implementations, the server-side TV application 186 may cause the server computer 106 to send or transmit the media content recommendation stream to the media adapter 107 by way of network 150. The media adapter 107 may cause the display of the media content recommendation stream in the UI 126.

In some implementations, multiple media content providers may source the plurality of recommended and categorized media content items included in a media content recommendation stream. A user interface (e.g., the UI 124, the UI 126) may display an ordered sequence of selectable information items on a display of a computing device (e.g., the mobile computing device display 122, the display 125 of the network-connected display device 104, respectively). For each selectable information item, the media content recommendation stream may include a respective command to be executed in response to a selection of the respective selectable information item. The selectable information items may be organized by category into clusters and arranged without referring to the content provider for the media content. For example, two selectable information items associated with media content provided by distinct media content providers may be placed adjacent to each other in a cluster.

In some implementations, an application on a mobile computing device of a user may include the functionality to provide personalized fitness content recommendations. In some implementations, the application may be a standalone application. In some implementations, the application may be a TV application (e.g., the TV application 110). In some implementations, an application on a server computer (e.g., the server-side TV application 186) may include the functionality to provide personalized fitness content recommendations (e.g., the server-side TV application 186). In these implementations, the server computer may send or transmit a media content recommendation stream to a media adapter for displaying in a user interface on a network-connected display device. In these implementations, the server computer may send or transmit a media content recommendation stream by way of a network to a smart television for displaying in a user interface of the smart television.

FIG. 4 illustrates a flowchart 400 depicting example operations of gathering fitness and health data, setting fitness goals, and providing personalized fitness recommendations. Although the flowchart 400 of FIG. 4 illustrates the operations in sequential order, it will be appreciated that this is merely an example, and that additional or alternative operations may be included. Further, operations of FIG. 4 and related operations may be executed in a different order than that shown, or in a parallel or overlapping fashion. The operations may define a computer-implemented method. Although the flowchart 400 is described with reference to the system 100 of FIG. 1B, the flowchart 400 may be executed according to any of the figures discussed herein. In some examples, the operations of the flowchart 400 are executed by the mobile computing device 102.

Operation 410 includes rendering, by a television application executing on a computing device, a first user interface that provides a fitness goal setup option associated with an account of a user of the television application. For example, referring to FIGS. 2A-B, the TV application 110 may receive an indication of the selection of the fitness goal setup option 206. The TV application 110 may generate the first user interface 220 for display in the UI 124.

Operation 420 includes receiving, by the television application, fitness goal criteria for the fitness goal setup option. For example, referring to FIG. 2C, the user may interact with the sliding buttons 218a-h for the fitness goal criteria 216a-h to select or deselect (not select) the associated fitness goal criterion.

Operation 430 includes sending, by the computing device, the fitness goal criteria to a server computer for use in generating a media content recommendation directed towards achieving the fitness goal criteria. For example, the mobile computing device 102 may send the fitness goal criteria to the server computer 106 for storage in the fitness goal criteria repository 132. The AI module 194 may use the fitness goal criteria along with other information and data associated with the user (e.g., fitness data, record of media content viewing) to determine media content recommendations for the user.

Operation 440 includes receiving a sequence of selectable information items that correspond to media content items sourced by a plurality of different media content providers based on the media content recommendation. For example, the server computer 106 may facilitate the sourcing of the recommended media content from the media content providers 108 to the mobile computing device 102.

Operation 450 includes displaying, by the television application, a second user interface that includes the sequence of selectable information items. For example, referring to FIG. 2C, the TV application 110 may generate the second user interface 230.

In some examples, the techniques described herein relate to a method including: rendering, by a television application executing on a computing device, a first user interface that provides a fitness goal setup option associated with an account of a user of the television application; receiving, by the television application, fitness goal criteria for the fitness goal setup option; sending, by the computing device, the fitness goal criteria to a server computer for use in generating a media content recommendation directed towards achieving the fitness goal criteria; receiving a sequence of selectable information items that correspond to media content items sourced by a plurality of different media content providers based on the media content recommendation; and displaying, by the television application, a second user interface that includes the sequence of selectable information items.

In some examples, the techniques described herein relate to a method, further including receiving, by the computing device, information and data from a wellness device, the information and data providing an indication of at least one of a physical activity of the user or a wellness state of the user.

In some examples, the techniques described herein relate to a method, further including, in response to receiving the information and data from the wellness device, providing the information and data received from the wellness device to the server computer for use along with the fitness goal criteria in generating the media content recommendation directed towards achieving the fitness goal criteria.

In some examples, the techniques described herein relate to a method, wherein the method further includes: receiving updated information and data from the wellness device; and in response to receiving the updated information and data from the wellness device, providing the updated information and data received from the wellness device to the server computer for use along with the fitness goal criteria in generating an updated media content recommendation directed towards achieving the fitness goal criteria.

In some examples, the techniques described herein relate to a method, wherein the media content items are classified into at least one category based on the fitness goal criteria; and wherein displaying the second user interface that includes the sequence of selectable information items includes displaying the sequence of selectable information items for the media content recommendation classified into the at least one category in a cluster.

In some examples, the techniques described herein relate to a method, wherein the television application includes a plurality of tabbed interfaces; and wherein the method further includes: presenting the first user interface in a first tab of the plurality of tabbed interfaces; and presenting the second user interface in a second tab of the plurality of tabbed interfaces.

In some examples, the techniques described herein relate to a non-transitory computer-readable medium storing executable instructions that when executed by at least one processor of a server computer cause the at least one processor to execute operations, the operations including: receiving, by the server computer and from a mobile computing device, fitness goal criteria associated with an account of a user of a television application; receiving information and data indicative of at least one of a physical activity of the user or a wellness state of the user; generating a media content recommendation directed towards achieving the fitness goal criteria based on the data indicative of at least one of the physical activity of the user or the wellness state of the user, or a combination thereof; selecting media content items from a plurality of media content providers based on the media content recommendation; and sending a sequence of selectable information items that correspond to the media content items for displaying in a user interface of the television application.

In some examples, the techniques described herein relate to a non-transitory computer-readable medium, further including providing the information and data indicative of the at least one of the physical activity of the user or the wellness state of the user as training data to an artificial intelligence module.

In some examples, the techniques described herein relate to a non-transitory computer-readable medium, wherein generating the media content recommendation directed towards achieving the fitness goal criteria includes utilizing the artificial intelligence module.

In some examples, the techniques described herein relate to a non-transitory computer-readable medium, wherein sending the sequence of selectable information items that correspond to the media content items for displaying in the user interface of the television application further includes: categorizing the media content items into a plurality of categories; and for each category, sending a sequence of selectable information items that correspond to the media content items in a respective category for display in a respective cluster in the user interface of the television application.

In some examples, the techniques described herein relate to a non-transitory computer-readable medium, wherein the plurality of categories are related to plans and activities for achieving the fitness goal criteria.

In some examples, the techniques described herein relate to a non-transitory computer-readable medium, wherein the information and data indicative of at least one of the physical activity of the user or the wellness state of the user are first information and data; wherein the media content recommendation is a first media content recommendation; wherein the media content items are first media content items; wherein the sequence of selectable information items that correspond to the media content items are a first sequence of selectable information items that correspond to the first media content items; and wherein the operations further include: receiving second information and data indicative of at least one of the physical activity of the user or the wellness state of the user; generating a second media content recommendation directed towards achieving the fitness goal criteria based on the second information and data indicative of at least one of the physical activity of the user or the wellness state of the user; selecting second media content items from the plurality of media content providers based on the second media content recommendation; and providing a second sequence of selectable information items that correspond to the second media content items for displaying in the user interface of the television application.

In some examples, the techniques described herein relate to a non-transitory computer-readable medium, further including determining whether the second information and data indicative of at least one of the physical activity of the user or the wellness state of the user indicates an improvement in achieving the fitness goal criteria.

In some examples, the techniques described herein relate to a non-transitory computer-readable medium, further including, based on determining that the second information and data indicative of at least one of the physical activity of the user or the wellness state of the user indicates an improvement in achieving the fitness goal criteria, providing the second information and data indicative of the at least one of the physical activity of the user or the wellness state of the user and the second media content items as training data to an artificial intelligence module.

In some examples, the techniques described herein relate to a system including: at least one processor; and a non-transitory computer-readable medium storing executable instructions that execute a television application on a computing device, the television application configured to: render a first user interface that provides a fitness goal setup option associated with an account of a user of the television application; receive fitness goal criteria for the fitness goal setup option; cause the computing device to send the fitness goal criteria to a server computer for use in generating a media content recommendation directed towards achieving the fitness goal criteria; receive a sequence of selectable information items that correspond to media content items sourced by a plurality of different media content providers based on the media content recommendation; and display a second user interface that includes the sequence of selectable information items.

In some examples, the techniques described herein relate to a system, wherein the television application is further configured to receive information and data from a wellness device, the information and data providing an indication of at least one of a physical activity of the user or a wellness state of the user.

In some examples, the techniques described herein relate to a system, wherein the television application is further configured to, in response to receiving the information and data from the wellness device, provide the information and data received from the wellness device to the server computer for use along with the fitness goal criteria in generating the media content recommendation directed towards achieving the fitness goal criteria.

In some examples, the techniques described herein relate to a system, wherein the television application is further configured to: receive updated information and data from the wellness device; and in response to receiving the updated information and data from the wellness device, provide the updated information and data received from the wellness device to the server computer for use along with the fitness goal criteria in generating an updated media content recommendation directed towards achieving the fitness goal criteria.

In some examples, the techniques described herein relate to a system, wherein the media content items are classified into at least one category based on the fitness goal criteria; and wherein displaying the second user interface that includes the sequence of selectable information items includes displaying the sequence of selectable information items for the media content recommendation classified into the at least one category in a cluster.

In some examples, the techniques described herein relate to a system, wherein the television application includes a plurality of tabbed interfaces; and wherein the television application is further configured to: present the first user interface in a first tab of the plurality of tabbed interfaces; and present the second user interface in a second tab of the plurality of tabbed interfaces.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a non-transitory machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or non-transitory medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

In this specification and the appended claims, the singular forms “a,” “an” and “the” do not exclude the plural reference unless the context clearly dictates otherwise. Further, conjunctions such as “and,” “or,” and “and/or” are inclusive unless the context clearly dictates otherwise. For example, “A and/or B” includes A alone, B alone, and A with B. Further, connecting lines or connectors shown in the various figures presented are intended to represent example functional relationships and/or physical or logical couplings between the various elements. Many alternative or additional functional relationships, physical connections or logical connections may be present in a practical device.

Terms such as, but not limited to, approximately, substantially, generally, etc. are used herein to indicate that a precise value or range thereof is not required and need not be specified. As used herein, the terms discussed above will have ready and instant meaning to one of ordinary skill in the art.

Moreover, use of terms such as up, down, top, bottom, side, end, front, back, etc. herein are used with reference to a currently considered or illustrated orientation. If they are considered with respect to another orientation, it should be understood that such terms must be correspondingly modified.

Further, in this specification and the appended claims, the singular forms “a,” “an” and “the” do not exclude the plural reference unless the context clearly dictates otherwise. Moreover, conjunctions such as “and,” “or,” and “and/or” are inclusive unless the context clearly dictates otherwise. For example, “A and/or B” includes A alone, B alone, and A with B.

Although example methods, apparatuses and articles of manufacture have been described herein, the scope of coverage of this patent is not limited thereto. It is to be understood that terminology employed herein is for the purpose of describing particular aspects and is not intended to be limiting. On the contrary, this patent covers all methods, apparatus and articles of manufacture fairly falling within the scope of the claims of this patent.

Further to the descriptions above, a user may be provided with controls allowing the user to make an election as to both if and when systems, programs, or features described herein may enable collection of user information (e.g., a user's preferences, a user's current location, a user's credentials, etc.), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

What is claimed is:

1. A method comprising:
rendering, by a television application executing on a computing device, a first user interface that provides a fitness goal setup option associated with an account of a user of the television application;
receiving, by the television application, fitness goal criteria for the fitness goal setup option;
sending, by the computing device, the fitness goal criteria to a server computer for use in generating a media content recommendation directed towards achieving the fitness goal criteria;
receiving a sequence of selectable information items that correspond to media content items sourced by a plurality of different media content providers based on the media content recommendation; and
displaying, by the television application, a second user interface that includes the sequence of selectable information items.

2. The method of claim 1, further comprising receiving, by the computing device, information and data from a wellness device, the information and data providing an indication of at least one of a physical activity of the user or a wellness state of the user.

3. The method of claim 2, further comprising, in response to receiving the information and data from the wellness device, providing the information and data received from the wellness device to the server computer for use along with the fitness goal criteria in generating the media content recommendation directed towards achieving the fitness goal criteria.

4. The method of claim 3, wherein the method further comprises:
receiving updated information and data from the wellness device; and
in response to receiving the updated information and data from the wellness device, providing the updated information and data received from the wellness device to the server computer for use along with the fitness goal criteria in generating an updated media content recommendation directed towards achieving the fitness goal criteria.

5. The method of claim 1,
wherein the media content items are classified into at least one category based on the fitness goal criteria; and
wherein displaying the second user interface that includes the sequence of selectable information items comprises displaying the sequence of selectable information items for the media content recommendation classified into the at least one category in a cluster.

6. The method of claim 1,
wherein the television application includes a plurality of tabbed interfaces; and
wherein the method further comprises:
presenting the first user interface in a first tab of the plurality of tabbed interfaces; and
presenting the second user interface in a second tab of the plurality of tabbed interfaces.

7. A non-transitory computer-readable medium storing executable instructions that when executed by at least one processor of a server computer cause the at least one processor to execute operations, the operations comprising:
receiving, by the server computer and from a mobile computing device, fitness goal criteria associated with an account of a user of a television application;
receiving information and data indicative of at least one of a physical activity of the user or a wellness state of the user;
generating a media content recommendation directed towards achieving the fitness goal criteria based on the data indicative of at least one of the physical activity of the user or the wellness state of the user, or a combination thereof;
selecting media content items from a plurality of media content providers based on the media content recommendation; and
sending a sequence of selectable information items that correspond to the media content items for displaying in a user interface of the television application.

8. The non-transitory computer-readable medium of claim 7, further comprising providing the information and data indicative of the at least one of the physical activity of the user or the wellness state of the user as training data to an artificial intelligence module.

9. The non-transitory computer-readable medium of claim 8, wherein generating the media content recommendation directed towards achieving the fitness goal criteria comprises utilizing the artificial intelligence module.

10. The non-transitory computer-readable medium of claim 7, wherein sending the sequence of selectable information items that correspond to the media content items for displaying in the user interface of the television application further comprises:
categorizing the media content items into a plurality of categories; and
for each category, sending a sequence of selectable information items that correspond to the media content items in a respective category for display in a respective cluster in the user interface of the television application.

11. The non-transitory computer-readable medium of claim 10, wherein the plurality of categories are related to plans and activities for achieving the fitness goal criteria.

12. The non-transitory computer-readable medium of claim 7,
wherein the information and data indicative of at least one of the physical activity of the user or the wellness state of the user are first information and data;
wherein the media content recommendation is a first media content recommendation;
wherein the media content items are first media content items;
wherein the sequence of selectable information items that correspond to the media content items are a first sequence of selectable information items that correspond to the first media content items; and
wherein the operations further comprise:
receiving second information and data indicative of at least one of the physical activity of the user or the wellness state of the user;
generating a second media content recommendation directed towards achieving the fitness goal criteria based on the second information and data indicative of at least one of the physical activity of the user or the wellness state of the user;
selecting second media content items from the plurality of media content providers based on the second media content recommendation; and
providing a second sequence of selectable information items that correspond to the second media content items for displaying in the user interface of the television application.

13. The non-transitory computer-readable medium of claim 12, further comprising determining whether the second information and data indicative of at least one of the physical activity of the user or the wellness state of the user indicates an improvement in achieving the fitness goal criteria.

14. The non-transitory computer-readable medium of claim 13, further comprising, based on determining that the second information and data indicative of at least one of the physical activity of the user or the wellness state of the user indicates an improvement in achieving the fitness goal criteria, providing the second information and data indicative of the at least one of the physical activity of the user or the wellness state of the user and the second media content items as training data to an artificial intelligence module.

15. A system comprising:
at least one processor; and
a non-transitory computer-readable medium storing executable instructions that execute a television application on a computing device, the television application configured to:
render a first user interface that provides a fitness goal setup option associated with an account of a user of the television application;
receive fitness goal criteria for the fitness goal setup option;
cause the computing device to send the fitness goal criteria to a server computer for use in generating a media content recommendation directed towards achieving the fitness goal criteria;
receive a sequence of selectable information items that correspond to media content items sourced by a plurality of different media content providers based on the media content recommendation; and
display a second user interface that includes the sequence of selectable information items.

16. The system of claim 15, wherein the television application is further configured to receive information and data from a wellness device, the information and data providing an indication of at least one of a physical activity of the user or a wellness state of the user.

17. The system of claim 16, wherein the television application is further configured to, in response to receiving the information and data from the wellness device, provide the information and data received from the wellness device to the server computer for use along with the fitness goal criteria in generating the media content recommendation directed towards achieving the fitness goal criteria.

18. The system of claim 17, wherein the television application is further configured to:
receive updated information and data from the wellness device; and
in response to receiving the updated information and data from the wellness device, provide the updated information and data received from the wellness device to the server computer for use along with the fitness goal criteria in generating an updated media content recommendation directed towards achieving the fitness goal criteria.

19. The system of claim 15,
wherein the media content items are classified into at least one category based on the fitness goal criteria; and
wherein displaying the second user interface that includes the sequence of selectable information items comprises displaying the sequence of selectable information items for the media content recommendation classified into the at least one category in a cluster.

20. The system of claim 15,
wherein the television application includes a plurality of tabbed interfaces; and
wherein the television application is further configured to:
present the first user interface in a first tab of the plurality of tabbed interfaces; and
present the second user interface in a second tab of the plurality of tabbed interfaces.
